(19) United States
(12) Patent Application Publication
(10) Pub. No.: US 2011/0200984 A1
(43) Pub. Date: Aug. 18, 2011

(54) USING NUCLEIC ACIDS FOR CLINICAL MICROBIOLOGY TESTING

(75) Inventor: Stephen O'Hara, (US)

(73) Assignee: 3M Innovative Properties Company

(21) Appl. No.: 13/096,518

(22) Filed: Apr. 28, 2011

Related U.S. Application Data
(63) Continuation of application No. 10/576,631, filed on Apr. 16, 2007, now abandoned.

(30) Foreign Application Priority Data
Oct. 22, 2003 (GB) GB0324650.1

(51) Int. Cl. C12Q 1/70; C12Q 1/68

(52) U.S. Cl. 435/5; 435/6.15; 435/6.12; 435/6.11

(57) ABSTRACT

A process for analysing a biological sample, comprising the steps of: (a) identifying a micro-organism present within the sample; and (b) determining the effect of one or more antimicrobial(s) on a micro-organism from the sample, wherein steps (a) and (b) are performed by analysing the micro-organism's nucleic acid. Steps (a) and (b) will generally occur in that order, but they may take place concurrently. The steps may advantageously be performed within a single apparatus. Conveniently, the nucleic acid analyte used for step (a) is the same as that used in step (b) e.g. the same PCR amplicon. A micro-organism's nucleic acids can thus be used both to identify the presence of the micro-organism within a sample and then to assess the effect of antimicrobials on its growth.

### CROSS REFERENCE TO RELATED APPLICATIONS

This application is a continuation of U.S. patent application Ser. No. 10/576,631, filed Apr. 16, 2007, which was a national stage filing under 35 U.S.C. 371 of PCT/GB2004/004494, filed Oct. 22, 2004, which claims priority to GB0324650.1, filed Oct. 22, 2003, the disclosure of which is incorporated by reference in their entirety herein.

All documents cited herein are incorporated by reference in their entirety.

### TECHNICAL FIELD

This invention is in the field of clinical diagnostic microbiology.

### BACKGROUND ART

Infectious diseases are the second largest cause of death worldwide and are the biggest killers of children. Antibiotics are generally useful for treating bacterial infections, but antibiotic resistance is on the increase.

When a patient has a bacterial infection, a physician needs to know how best to treat it, and the best treatment will depend on the antibiotic susceptibility of the bacterium in question. Identification of the bacterium and of its antibiotic susceptibility can be performed in clinical laboratories but, despite the high global human and financial cost of infectious diseases, many tests for pathogenic bacteria still use the labour-intensive methods developed by Pasteur in the 19th century. In these tests, bacteria from the patient are grown on plates in specialised culture media until they reach sufficient numbers to be seen by the human eye. This culture step takes 24-48 hours and, once the bacteria have been identified, it takes a further 24 hours before antibiotic susceptibility can be determined.

From taking a patient sample to identifying an appropriate antimicrobial therapy therefore takes 2 to 3 days. Current infectious disease diagnostics therefore have little impact on patient management. As a physician cannot afford to wait 3 days before starting treatment, patients are subjected to ‘best guess’ antimicrobial therapy, and this typically involves expensive broad-spectrum antimicrobials which may be unnecessary or inappropriate. Hospital stays are increased in length, leading to higher costs, and there is also an increased risk that antimicrobial resistance will develop.

It is an object of the invention to provide improvements in clinical diagnostic microbiology. In particular, it is an object to provide systems and methods for both rapid identification and rapid antimicrobial sensitivity testing of clinically-important bacteria.

### DISCLOSURE OF THE INVENTION

It has now been realised that the micro-organism's nucleic acids can be used both to identify the presence of the micro-organism within a sample and then to assess the effect of antimicrobials on its growth. Thus the invention provides a process for analysing a biological sample, comprising the steps of: (a) identifying a micro-organism present within the sample; and (b) determining the effect of one or more antimicrobial(s) on a micro-organism from the sample, wherein steps (a) and (b) are performed by analysing the micro-organism's nucleic acid.

Steps (a) and (b) will generally occur in that order, but they may take place concurrently. The steps may advantageously be performed within a single apparatus. Conveniently, the nucleic acid analyte used for step (a) is the same as that used in step (b) e.g. the same PCR amplicon.

The nucleic acid to be analysed may be DNA or RNA.

### Micro-Organism Identification

Micro-organism identification was traditionally based on phenotype e.g. on morphology, growth characteristics, etc. Genotype-based techniques are now available [1] which allow a micro-organism to be identified on the basis of nucleic acid sequence (e.g. use of PCR has been widely described [e.g. refs. 2 to 7]), and these techniques are rapid, sensitive and specific.

There are four main techniques for identifying a micro-organism based on its nucleic acid: (i) nucleic acid hybridisation; (ii) nucleic acid sequencing; (iii) nucleic acid separation; and (iv) amplicon detection. Sequence data for the micro-organism is absolutely required for the second technique and is usually used for the first and fourth techniques, but this is not always necessary (e.g. hybridisation probes of unknown sequence can be used, and random amplification primers can be used).

Hybridisation techniques involve the use of a probe. The probe can hybridise to a target single-stranded nucleic acid and the hybridisation event can be detected. Typically, therefore, the probe is labelled e.g. with a fluorescent label (as in FISH [8]), a radioactive label (e.g. a Southern blot), etc. In an alternative arrangement, the probe includes half of a binding pair, and the second half of the pair carries the label e.g. the probe includes a biotin tag which is detected by a labelled streptavidin, or the probe contains a tag sequence to which a further labelled nucleic acid is hybridised. The branched DNA (bDNA) assay works in this way [9]. Molecular beacon probes [10] may be used.

The probe may be immobilised on a solid support e.g. on a filter, a membrane, a bead, a glass slide, a gene chip [11], a metal (e.g. gold) substrate (e.g. a film) [12], etc. Immobilisation helps separation of hybridised and non-hybridised nucleic acids.

Hybridisation probes are already widely available for identifying many micro-organism of interest [e.g. see refs. 13-15]. New probes can be designed based on genomic sequence information [16], with comparative genomics of related bacteria being particularly useful for designing specific probes.

The probe may hybridise to any suitable region of the micro-organism genome (including the chromosome and extra-chromosomal material, such as plasmids in bacteria and fungi). Many probes for micro-organisms are based on rRNA or rDNA sequences [e.g. refs. 17 & 18], but other sequences (e.g. protein-coding sequences) may also be used. For example, ref 19 describes an array for distinguishing 59 types of methanotrophs based on their methane monooxygenase (pmoA) genes.

The probe may be made of DNA, RNA, or a modified form of either e.g. PNA [20], etc.

Sequencing techniques involve the determination of at least part of a micro-organism's genome sequence. For example, the sequence of the gene encoding a bacteria's 16S rRNA can be determined and the micro-organism can be identified by checking that sequence against known sequences. The use of 16S sequencing for pathogen identification in the clinical laboratory is reviewed in ref 21.

Nucleic acid separation can be used to identify a micro-organism using techniques such as restriction fragment length polymorphism (RFLP) [22] or amplified rDNA restriction analysis (ARDRA) [23].

Amplicon detection techniques involve amplification of a micro-organism's nucleic acid from within a sample and the amplified material (the amplicon) is then detected [24]. The amplicon can be detected by any suitable technique e.g. by detecting an amplicon of a specific length on an agarose gel, by melting curves, etc. Amplicon detection can be used in conjunction with the hybridisation, separation and sequencing techniques described above e.g. a probe can be hybridised with the amplicon, rather than with the micro-organism's own nucleic acid, or the amplicon can be sequenced, or RFLP can be performed on an amplicon [25, 26], etc.

Whichever of these (or other) techniques is chosen, the target of the technique (e.g. the hybridisation target sequence, or the amplicon) will be specific to target micro-organisms and will advantageously be a gene involved in antimicrobial resistance [27-29]. Thus, in step (b), the invention can yield genotypic and phenotypic information about antimicrobial resistance. Suitable target sequences include the genes for methicillin resistance (mecA), vancomycin resistance (vanA, vanE, vanC), tetracycline resistance [30], aminoglycoside resistance (aacA, aphD), tetracycline resistance (tetK, tetM), macrolide lincosamide streptogramin B resistance (ermA, ermC), ciprofloxacin resistance (gyrA, gyrE, parC), beta-lactam resistance (blaTEM, bla(SHV), blaOXA-1), etc.

### Antimicrobial Testing

The invention involves testing one or more antimicrobials on micro-organisms from a patient sample. The goal of the test is to see whether a micro-organism is able to grow when cultured with a particular antimicrobial, or whether its growth is inhibited. Antimicrobials which inhibit growth will be suitable for treating the patient from whom the sample was derived. According to the invention, microbial numbers are assessed by measuring nucleic acid content in a sample—when an antimicrobial is ineffective then nucleic acid content will increase as micro-organisms divide.

In general, the detection of nucleic acid for assessing micro-organism numbers can use the same techniques as described above for micro-organism detection. The use of a common technique for steps (a) and (b) simplifies clinical microbiology and is a significant advantage of the invention.

As it is necessary to determine whether nucleic acid levels are rising, falling or remaining static over time, it is preferred to use a quantitative technique. A preferred technique is quantitative PCR. Real-time techniques are also preferred [31]. Real-time multiplex quantitative PCR using molecular beacons is disclosed in reference 32 for detection of methicillin resistance in Staphylococcus aureus.

The use of nucleic acid content as a measure of antimicrobial efficacy is known [refs. 33-36], with quantitative PCR on the LightCycler™ instrument being the method of choice. These methods are very sensitive and are well suited to precise measurement of copy numbers within a sample.

Using the same technique in steps (a) and (b) for analysing nucleic acid is advantageous for two main reasons. First, it simplifies the instrumentation which is needed for the overall analysis. Second, the results obtained in step (a) can be used as a “time zero” value for step (b). As well as using the same technique in step (a) and (b), it is preferred to use the same nucleic acid analyte in both steps e.g. to use the same hybridisation probe, the same target for sequencing, the same target RFLP, the same amplicon (e.g. by using the same primers and, optionally, probe), etc.

To minimise waste, it is preferred to base antimicrobial testing in step (b) on the results of micro-organism identification from step (a). For example, if a test in step (a) reveals that a particular micro-organism is not present in a sample then it will not be necessary to perform antimicrobial testing for that micro-organism in step (b) i.e. antimicrobial testing need only be performed against micro-organisms which have given a positive identification result in step (a). In some situations, negative identification results may mean that antimicrobial testing is not even performed (e.g. if the sample is tested to identify the presence only of MRSA, but the bacterium is absent, the result of the test is simply ‘negative’ and there is no need to test antimicrobial susceptibility).

A second way of using the results from step (a) to educate antimicrobial testing in step (b) is to select particular panels of antimicrobials for testing. If a penicillin-resistant and amoxycillin-resistant bacterium is identified, for example, the antimicrobials which are subsequently tested against that bacterium can be adapted accordingly.

A preferred antimicrobial testing technique is AST (antimicrobial susceptibility testing [e.g. refs. 37 to 39]). Essentially, this technique involves incubating a micro-organism in the presence of a number of different antimicrobials in order to determine which antimicrobial(s) can inhibit the growth of the micro-organism and thus be suitable for patient treatment. Another antimicrobial test which may be performed is the generation of a killing curve, in which the effect of an antimicrobial at a given concentration is followed over time.

If antimicrobials are tested at various concentrations, the technique can be used to identify minimum inhibitory concentration (MIC) values for antimicrobials (i.e. the lowest concentration of a particular antimicrobial which can inhibit the growth of a given micro-organism) or minimum bactericidal concentration (MBC) values (i.e. the lowest concentration which can kill a given micro-organism).

According to the invention, a plurality of antimicrobials can be tested (e.g. 2, 3, 4, 5, 6, 7, 8, 9, 10, 15, 20 or more). Furthermore, a plurality of concentrations of each antimicrobial can preferably be tested (e.g. 2, 3, 4, 5, 6, 7, 8, 9, 10, 15, 20 or more), and preferably between 4 and 8 (e.g. 6). It is preferred to test a range of concentrations in the range from 0.05 to 50 mg/ml, more preferably a range from 0.125 to 16 mg/l. The range preferably spans the known break-point for an antimicrobial.

For a single AST, MIC or MBC test, the process of the invention may involve: adding an antimicrobial at a pre-determined concentration to a sample; incubating the sample in the presence of the antimicrobial for a pre-determined time period (e.g. a period which would allow ˜2 logs of growth in the absence of antimicrobial); and assessing the number of micro-organisms in the sample at the end of said time period by use of DNA detection.

For a complete AST test, the process of the invention may involve: adding n different antimicrobials at pre-determined concentrations to n different sub-samples; incubating the sub-samples in the presence of the antimicrobials for pre-determined time periods; and assessing the numbers of micro-organisms in the sub-samples at the end of said time periods by use of DNA detection.

For a complete MIC or MBC test, the process of the invention may involve: adding an antimicrobial at n pre-determined concentrations to n different sub-samples; incubating the sub-samples in the presence of the antimicrobial for pre-determined time periods; and assessing the numbers of micro-organisms in the sub-samples at the end of said time periods by use of DNA detection.

For killing curve testing, the process of the invention may involve: adding an antimicrobial at a pre-determined concentration to a sub-sample; incubating the sub-sample in the presence of the antimicrobial for a pre-determined time period; and assessing the number of micro-organisms in the sub-sample at a plurality of time points within said time period by use of DNA detection.

These tests will typically also include a step of determining the number of micro-organisms in a sub-sample at time zero. Advantageously this value is taken from tests performed in step (a).

The process of the invention may further comprise the step of using the results of the antimicrobial testing step to calculate a MIC and/or MBC value for a given micro-organism in a patient sample. MIC values may be presented as true MICs, abridged MICs, or calculated MICs.

Antimicrobial testing will typically be accompanied by a control analysis in which a micro-organism is incubated in the absence of antimicrobials. In addition, the process of the invention may include control tests. Typical negative controls could be to try step (a) or (b) on basic culture medium, etc.

In general, assessment of micro-organism numbers in a sub-sample taken at a specific time will not be performed immediately. A typical process will thus require the inhibition of further growth in a sub-sample once an assessment is to be made. Further growth can be inhibited by addition of “stop solution” such as an azide, by cooling or rapid freezing, by lysis, etc.

Incubation steps preferably take place at a predetermined temperature e.g. at 37±2° C. Higher temperatures may be used if desired e.g. at 41° C. the doubling time of E. coli is 7 minutes, compared to 20 minutes at 37° C., so higher temperatures can accelerate analysis. Higher temperatures are also useful for some slow-growing organisms. The temperature preferences of different bacteria are well known in microbiology and temperatures used in the invention can be modified accordingly.

If step (b) is performed on total DNA extracts from samples (or sub-samples thereof) then there is an additional benefit. If total DNA is seen to rise even though DNA from all micro-organisms of interest is declining then the user is alerted to the presence of other organisms in the sample.

### Micro-Organism Separation

If the techniques used in step (a) or (b) are micro-organism-specific then different micro-organisms can be tested in parallel. Thus an extract of total DNA for a single aliquot can be analysed in parallel to determine what micro-organisms are present. Even in a mixed culture, therefore, the growth of micro-organisms X and Y can be followed in parallel e.g. it is possible to see that micro-organism X continues to proliferate whilst micro-organism Y is dying. Differential labelling of probes can be used to assist in parallel analysis.

In some embodiments of the invention, however, it is helpful to separate different micro-organisms from each other. This separation can take place before step (a), before step (b), or after step (b).

Thus the process might involve an initial step of: extracting a specific micro-organism. The extracted micro-organism can then be subjected to step (a) in order to identify it. As the extraction step was specific, step (a) can then be general. For example, if an initial extraction step is MRSA-specific then step (a) need not be MRSA-specific e.g. it can use an identification technique which gives a positive result for all bacteria. It is possible to use a specific identification technique after a specific extraction step, provided that the two are matched e.g. it is not helpful to use a MRSA-specific identification technique if the extraction technique was specific to Streptococcus pneumoniae.

If an extraction is not performed prior to step (a) then the identification technique should be specific. For example, if a sample is treated to obtain its total DNA then a particular micro-organism (e.g. MRSA) can be identified only by using a technique specific to that micro-organism (e.g. primers and/or probes specific to MRSA).

In general, therefore, either the pre-extraction step or the identification step will be specific to a micro-organism. In some situations, however, the invention can be performed without pre-extraction and without using a specific assay in step (a). Such a process identifies that a sample contains micro-organism(s) and identifies whether it/they are susceptible to an antimicrobial, and this result may be satisfactory e.g. if it is known that a batch of samples is either sterile or is contaminated with a new MRSA strain then this result is adequate.

If step (a) has identified a specific micro-organism by the use of a specific identification, then a specific extraction can be performed prior to step (b). Advantageously the extraction will be based on the results of step (a) i.e. an extraction for organism X will not be performed if identification of organism X was negative in step (a).

To extract micro-organisms from a sample in order to give one or more extracted micro-organism samples, various techniques can be used. For specificity, extraction methods generally rely on immunochemistry, using an antibody for a micro-organism-specific antigen. A preferred technique for use with the invention is based on immunomagnetic separation (IMS) methods, in which magnetic particles (typically beads) are coupled to antibodies specific for antigens on the surface of micro-organisms of interest [refs. 40 & 41]. The antibodies interact with micro-organisms to form particle-micro-organism complexes. These complexes can then be separated by the use of magnets. Organisms may also be extracted by techniques such as: the use of flow cytometry of cell sorting based on fluorescent labelling (e.g. FACS); differential filtration (e.g. based on physical or chemical characteristics of the cell, membrane affinity, tunable membranes, etc.); dielectrophoresis; capture based on cell-surface molecules such as ligand or lectin based capture; non-antibody receptors, such as recombinant phages or other combinatorial peptides; etc. In general, therefore, any physical, immunological or chemical means of extraction can be used, and the choice of extraction technique will depend on factors such as cost, convenience, sample type (e.g. some techniques are better for blood than others), desired specificity, etc.

Where IMS is used for extraction, the process of the invention may involve one or more of the following steps: (i) mixing immunomagnetic particles with a sample, wherein the particles comprise 5 an antibody which specifically binds to a target micro-organism; (ii) allowing the sample to interact with the particles; (iii) placing the sample in a magnetic separator which causes the magnetic particles to separate from the sample; (iv) aspirating liquid from the sample to leave the particles, without removing bound micro-organisms; and (v) washing the particles with a solution e.g. to remove preservative, non-specific binding substances and loosely bound micro-organisms.

There are three principal routes by which extracted micro-organism samples can be prepared ready for use in steps (a) and/or (b): (1) one extraction is performed on a sample, to give an extracted micro-organism sample, which is then split to give a first extracted micro-organism (sub)-sample for use in step (a) and a second for use in step (b); (2) two separate extractions are performed on the same sample, to give a first extracted micro-organism sample for use in step (a) and a second for use in step (b); (3) a sample is divided, with a first sub-sample being subjected to extraction to give material for step (a) and a second sub-sample being subjected to extraction to give micro-organisms for step (b). It is preferred that the same route is used for each micro-organism to be extracted e.g. all micro-organisms are extracted via route (2), rather than using route (1) for streptococci and route (3) for meningococci, etc. [NB: although this passage refers to “two” extractions, it will be appreciated 20 that the figure “two” refers to the eventual fate of the extracted material, rather than implying that only “two” extractions can physically take place. Thus the “two” extractions in (2) could involve many more than two physical acts of extraction, and the patient sample could be divided in (3) to give many more than two sub-samples, but the extracted material may have a first fate (identification) or a second fate (antimicrobial testing).].

It is important that any extraction technique which is used should not kill the micro-organism, as antimicrobial testing must be performed on living micro-organisms in order to give a useful result.

Although step (a) can be performed on dead micro-organisms, using a lethal extraction technique for identification purposes and a non-lethal technique for susceptibility testing purposes is more complicated than using a common non-lethal technique for both purposes. Furthermore, the use of a non-lethal technique for extraction allows the extracted micro-organisms to be used for purposes other than antimicrobial testing.

It is also preferred that the extraction technique should not inhibit growth of the micro-organism as antimicrobial testing requires multiplying or growing micro-organisms, and reversing the growth inhibition before commencing identification is more complicated than is necessary.

Where micro-organism extraction is used in the process of the invention, a plurality of micro-organisms can be extracted from samples (e.g. 2, 3, 4, 5, 6, 7, 8, 9, 10, 15, 20 or more), although in some embodiments it may be desired to extract only a single organism. Different micro-organisms may be extracted together or simultaneously in parallel, but it is more typical to extract them separately in series, particularly where immunomagnetic methods are used.

It will be appreciated that a micro-organism can only be “extracted” if it is present in a sample. References to “extracting” a micro-organism should therefore be interpreted as referring to the potential to do so if the micro-organism is present. Thus the invention could be used to “extract” micro-organisms from a sample which contains no micro-organisms (e.g. a sterile sample), but none of the extraction steps performed on the sample would actually result in any micro-organisms being removed. Nevertheless, the sample has been subjected to micro-organism “extraction”.

### Nucleic Acid Amplification Techniques

Steps (a) and (b) will typically involve the use of nucleic acid amplification. As mentioned above, quantitative and/or real-time techniques are preferred.

Nucleic acid amplification techniques (NAATs) which can be used include thermal cycling techniques as well as isothermal techniques e.g. the polymerase chain reaction (PCR), reverse transcriptase PCR (RT-PCR), the ligase chain reaction (LCR), rolling-circle amplification (RCA) [42], boomerang DNA amplification (BDA) [43], transcription-mediated amplification (TMA) [44], the Qb replicase system, the repair chain reaction (RCR), self-sustaining sequence replication (3SR), the strand displacement assay (SDA), nucleic acid sequence based amplification (NASBA), etc.

The skilled person can choose which technique to use based on their needs. Comparisons of LCR vs. PCR for Chlamydia trachomatis detection are given in references 45-49, a similar comparison for Mycobacterium tuberculosis diagnosis is seen in reference 50, and another LCR vs. PCR comparison is given in reference 51 for Neisseria gonorrhoeae. TMA and PCR diagnosis of C. trachomatis are compared in reference 52, LCR and TMA are compared in reference 53, and a 3-way comparison (TMA, PCR, SDA) is described in reference 54. PCR, nested PCR and RAPD-PCR are compared in reference 55 for detecting and typing Ureaplasma urealyticaum. TMA has been found to be more sensitive than PCR for detecting hepatitis C virus [56].

These amplification techniques generally involve the use of one or more pairs of primers which hybridise to opposite strands of a double-stranded target. One or both of these primers can be specific to a particular micro-organism if required, to ensure that the amplified sequence is produced only for intended micro-organisms. As an alternative, random or arbitrary primers can be used (e.g. as in the random amplification of polymorphic DNA (RAPD) technique [18]), with the amplified material then being further analysed to determine its origin. Sequence information can be used to design primers with a desired specificity [e.g. refs. 57-66]. Specificity can also be achieved via probes used to detect amplified sequences. Specificity can also be achieved by determining the length of an amplicon e.g. even where the same primers and probes are used.

Multiplex amplification and/or detection techniques [e.g. refs. 29 & 30] may conveniently be used. Thus nucleic acid from more than one microorganism can be amplified and/or detected in the same reaction. Different detectable labels can be used for different microorganisms. As an alternative to multiplexing, a sample can be split into sub-samples and separate microorganism-specific amplification reactions can be performed on each.

Melting curves can conveniently be used to measure nucleic acid concentrations. These allow different quantities of a single product to be compared over time, and also allow quantitative detection of different amplicons even in the same reaction mixture. Fluorometry is generally used.

Two ways of quantifying nucleic acids are: (a) assess the amount of nucleic acid after a set period of time or a set number of amplification cycles; (b) assess the period of time or number of amplification cycles to reach a given amount of nucleic acid.

### The Patient Sample

Specimens taken from patients (e.g. blood, stools, swabs, mucous, tissue, etc.) are generally not suitable for direct microbiological testing, and so have to be processed (e.g. liquefied, diluted, separated) to give samples for use with the invention. Some specimens, however, such as urine or cerebrospinal fluid can be used directly as samples with the invention, without processing. Where it is required, specimen processing will generally be performed prior to the process of the invention.

The term ‘patient sample’ therefore includes both material taken directly from a patient and material obtained by processing material taken directly from a patient (i.e. indirectly obtained from the patient, e.g. a blood culture). In some embodiments, it includes a micro-organism plate culture obtained from material taken from a patient. Advantageously, however, the invention avoids the prior need for such organism plate culture.

For some types of sample where micro-organism numbers are low (e.g. in swabs) a short incubation step to increase micro-organism numbers may advantageously be used for increasing sensitivity, but this is not essential. This incubation step will generally take place after micro-organism extraction.

The process of the invention can be performed on a patient sample or on material derived from a patient sample e.g. on aliquots of the sample, or on a culture of bacteria which were present in the sample, etc.

### Micro-Organisms

The invention can specifically extract and detect a plurality of different micro-organisms from within a patient sample. The degree of specificity in extraction/detection may depend on the needs of an individual user. Taking streptococci as an example: it may be desired to extract all streptococci in a sample; it may be desired to extract individual species (e.g. separate extraction of S. pneumoniae and S. agalactiae and S. mutans); it may be desired to extract particular serotypes (e.g. separate extraction of serotypes 6B, 14, 19, 23F of S. pneumoniae); or it may be desired to extract particular strains (e.g. separate extraction of penicillin-resistant and penicillin-sensitive strains of S. pneumoniae). At a higher level, it may be desired to extract all bacteria or all yeast, depending on the user's needs. At all of these levels, however, the extraction is “specific” in the sense that micro-organisms of interest are extracted but other micro-organisms are left in the sample. The same criteria apply for detection.

For example, reference 67 discloses a method for detecting Coxiella burnetii by specific hybridization of labelled DNA probes to rickettsial plasmid DNA sequences in clinical samples. Two types of probe are used in the methods—one which detects all C. burnetii strains, and one which can differentiate between organisms associated with chronic or acute disease. Similarly, reference 68 discloses oligonucleotide probes for quantitative analysis of methanotroph-specific 16S rRNA. Two probes target methanotrophs in the family Methylocystaceae as a group, and two other probes target, as a single group, a majority of the known methanotrophs belonging to the family Methylococcaceae, and the remaining probes target members of individual genera of the Methylococcaceae, including Methylobacter, Methylomonas, Methylomicrobium, Methylococcus, and Methylocaldum. Reference 69 discloses probes that target universal sequences (all bacteria), E. faecalis, Lachnospira multiparus, Fibrobacter succinogenes, Fibrobacter succinogenes, Fibrobacter intestinales, Bacteroides distasonis, Bacteroides vulgates, Bacteroides fragilis and Salmonella sp. Reference 60 discloses a PCR-based assay in which a single pair of PCR primers amplifies DNA from 65 strains of Bacteroides thetaiotamicron but not from any other bacterial species. The skilled person can thus choose which sort of probe to use based on their desired level of specificity.

The invention can be used with bacteria and/or fungi and/or parasites and/or viruses. The ability of the invention to deal with all of these various pathogens is a significant advantage of the invention Preferably, it can extract the following micro- organisms: Staphylococci, such as S. aureus (and more particularly methicillin-resistant S. aureus); Enterococci such as E. faecium and E. faecalis (and more particularly vancomycin-resistant E. faecalis); Streptococci such as S. pyogenes, S. pneumoniae (and more particularly penicillin-resistant S. pneumoniae), and S. agalactiae; Coliforms such as E. coli, Klebsiella species, Proteus species, and Enterobacter species; Enteric organisms like Salmonella species, Shigella species, and Campylobacter species; Neisseria species such as N. meningitidis, N. gonorrhoeae; yeasts, such as C. albicans; parasites such as P. falciparum, Leishmania; spirochaetes; schistosoma; specific bacterial pathogens such as Burkholderia cepacia, Bacillus anthracis, Clostridium botulinum, Yersinia pestis, Corynebacterium diphtheriae and Bordetella pertussis; herpes viruses, including herpes simplex virus (HSV); cytomegalovirus (CMV); retroviruses, including HIV, HTL V-I and HTL V-II; corona viruses, including SARS virus; and hepatitis viruses, including HA V, HBV and HCV. This list is not exhaustive, but serves to illustrate the wide range of disease-causing micro-organisms which can be analysed using the invention. However, in some embodiments the invention does not relate to intracellular pathogens.

Different organisms typically have different optimum growth conditions (media, aerobic/anaerobic, temperature, etc.). For example, streptococci grow well in Todd-Hewitt medium, whereas S. aureus prefers peptone. The invention may thus utilise a number of different conditions but, for simplicity, it is preferred to compromise by using ‘generic’ media e.g. BHI (brain heart infusion). Where the invention involves antimicrobial sensitivity testing of a specific organism, however, it requires a choice of conditions which allows some growth of that organism e.g. it may use a common medium for all micro-organisms except for one, which requires a specific medium. The choice of growth medium will ultimately depend on the choice of micro-organisms to be detected and such choices are familiar to workers in this field. The choice of growth medium may depend on geographical location e.g. the EU and USA have different standard methodologies.

### Antimicrobials

The invention involves the use of antimicrobials. The term “antimicrobial” refers to any substance (typically an organic compound) that can kill, or inhibit the growth of, a micro-organism. The term includes natural and synthetic compounds. It includes antibiotics, antimycotics and antivirals within its scope, with antibiotics being a preferred subset of antimicrobials.

Classes of antimicrobials which may be tested include beta-lactams, aminoglycosides, fluoroquinolones, sulfonamides, glycopeptides, carbapenems, azoles, oxazolidinones, macrolides, quinolones, tetracyclines, etc.

Typical antimicrobials for use with the invention are: penicillin, amoxycillin, ciprofloxacin, cephalothin, ampicillin, augmentin, linezolid, gentamicin, flucluxacillin, vancomycin, chloramphenicol, tetracycline, minocycline, sulfonamide, oxazolidinone, fluconazole, nitrofurantoin, trimethoprim, nalidixic acid, amphotericin, kanamycin, streptomycin, vidarabine, acyclovir, gancyclovir, AZT (zidovudine), 3TC (lamivudine), etc.

The invention may also be used to test the effect of mixtures of two or more antimicrobials. Testing combinations may identify positive or negative synergies between the antimicrobials against a particular extracted micro-organism.

Different antimicrobials typically have different activity profiles e.g. they may be slow- or quick-acting. Each antimicrobial test may therefore be different. As the invention involves the use of known antimicrobials, however, the invention can be adapted according to the profile of any particular antimicrobial. Antimicrobial testing will typically last between 15 minutes and 4 hours. Reading results at around 2 hours is generally convenient.

### General

The term “comprising” means “including” as well as “consisting” e.g. a composition “comprising” X may consist exclusively of X or may include something additional e.g. X+Y.

The term “about” in relation to a numerical value x means, for example, x±1 0%.

The word “substantially” does not exclude “completely” e.g. a composition which is “substantially free” from Y may be completely free from Y. Where necessary, the word “substantially” may be omitted from the definition of the invention.

The term “antibody” includes any suitable natural or artificial immunoglobulin or derivative thereof. In general, the antibody will comprise a Fv region which possesses specific antigen-binding activity. This includes, but is not limited to: whole immunoglobulins, antigen-binding immunoglobulin fragments (e.g. Fv, Fab, F(ab′)2 etc.), single-chain antibodies (e.g. scFv), oligobodies, chimeric antibodies, humanized antibodies, veneered antibodies, phage-displayed antibodies, etc.

The process of the invention may be performed on an apparatus as described in reference 70 i.e. an apparatus for microbiological analysis of a sample, comprising: (a) a micro-organism extractor, for extracting one or more micro-organism(s) from the patient sample to give one or more extracted micro-organism sample(s); (b) a temporary storage facility, for storing extracted micro-organism sample(s); (c) a micro-organism identifier, for identifying one or more specific micro-organism(s) within the patient sample and/or within the extracted micro-organism sample(s); (d) an antimicrobial tester, for determining the effect of one or more antimicrobial(s) on micro-organism(s) within extracted micro-organism sample(s) and/or within the patient sample; (e) a thermostatically-controlled incubator for incubating extracted micro-organism sample(s); (f) a timer; and (g) one or more sample routers, for routing: patient sample to the micro-organism extractor; extracted micro-organisms to the temporary storage facility; micro-organisms from the temporary storage facility, patient sample or extracted sample to the micro-organism identifier; and micro-organisms from the temporary storage facility to the antimicrobial tester.

### MODES FOR CARRYING OUT THE INVENTION

Specimens of blood are taken from hospitalised patients with unidentified microbial infections. Each specimen is split into ten aliquots. Bacteria in one the aliquots are lysed to release DNA and the absence/presence of S. aureus in the lysed aliquot is determined by PCR using the specific forward and reverse primers disclosed in Table I of reference 29, which give a 108bp amplicon.

For specimens where S. aureus infection is confirmed by this PCR, antibiotic testing is then performed in order to identify MIC values for gentamicin. Rather than use the disc diffusion method as used in reference 29, however, the effect of the antibiotic is assessed by quantitative PCR.

Six aliquots are used for the MIC testing, using six different antibiotic concentrations. A further aliquot serves as a negative control i.e. using zero antibiotic. The quantity of bacteria in each of these seven aliquots is assessed by quantitative PCR after the same period of incubation. The quantity of bacteria in a ninth aliquot is assessed at the beginning of the incubation period to give a reference value against which the quantity of bacteria in the seven test aliquots can be compared. The MIC value is determined by determining the lowest of the six antibiotic concentrations which results in inhibition of growth of S. aureus compared to the reference aliquot. The tenth aliquot is used as a positive control, and is grown in broth with no antimicrobials. Lack of growth with the positive control is a useful warning.

In an improved method, quantitative PCR is used for the initial S. aureus identification step, and the result of this assay is used as a time-zero value for the subsequent antibiotic susceptibility testing.

In a further experiment, each of the six test aliquots is incubated with a different antibiotic in order to determine the antibiotic susceptibility of S. aureus in the patient specimens.

Thus PCR can be used for both microbial identification and for testing the effect of antimicrobials.

It will be understood that the invention has been described by way of example only and modifications may be made whilst remaining within the scope and spirit of the invention.

### REFERENCES (The Contents of Which are Hereby Incorporated by Reference)

[1] Baselski (1996) Clin Lab Med. 16:49-60.

[2] Kassimi et al. (2002) J Virol Methods 101:197-206.

[3] Zhang & Weintraub (1998) J Clin Microbiol36:3545-3548.

[4] Dowd et al. (1998) Appl Environ Microbiol64:333-336.

[5] Li et al. (1996) J Clin Microbio/34:1903-1907.

[6] Shetab et al. (1998) J Clin Microbio/36:1729-1732.

[7] Homes et al. (1991) J Clin Microbiol29:2375-2379.

[8] Moter & Gobel (2000) J Microbial Methods. 41:85-112.

[9] Wilber (1997) Immunol Invest 26:9-13.

[10] Tyagi & Kramer FR (1996) Nature Biotechnol14:303-308.

[11] Anthony et al. (2001) Expert Rev Mol Diagn 1:30-38.

[12] Du et al. (2003) J Am Chem Soc 125:4012-4013.

[13] Macario & Macario (1990) Gene Probes for Bacteria. ISBN: 0124630006.

[14] Stackebrandt & Goodfellow (1994) Nucleic Acid Techniques in Bacterial Systematics. ISBN: 04 71929069.

[15] Landry & Fong (1985) Clin Lab Med 5:513-529.

[16] Giesendorf et al. (1996) Zentralbl Bakteriol283:417-430.

[17] Peplies et al. (2003) Appl Environ Microbio/69:1397-1407.

[18] Reiss et al. (1998) Med Mycol36 Supp\1:249-257.

[19] Bodrossy et al. (2002) Environ Microbiol5:566-582.

[20] Perry-O′Keefe et al. (2001) J Microbial Methods 47:281-292.

[21] Patel (2001) Mol Diagn 6:313-321.

[22] Nishimura et al. (1996) FEMS Microbial Lett 142:133-138.

[23] De Baere et al. (2002) BMC Microbiology 2:4.

[24] Ieven & Goossens (1997) Clin Microbiol Rev 10:242-256.

[25] Lee et al. (2002) Arch Virol147:1711-1720.

[26] Gaitanis et al. (2002) Clin Microbial Infect 8:162-173.

[27] Garcia & Vledma (2003) Clin Microbial Infect 9:349-359.

[28] Tan (2003) Expert Rev Mol Diagn 3:93-103.

[29] Martineau et al. (2000) J Antimicrob Chemother 46:527-534,

[30] Ng et al. (2001) Mol Cell Probes 15:209-215.

[31] Lyons et al. (2000) J Clin Microbio/38:2362-2365.

[32] Elsayed et al. (2003) Arch Pathol Lab Med 127:845-849.

[33] Rolain et al. (2002) Antimicrob Agents Chemother 46:2747-2751.

[34] Masse lot et al. (2003) Antimicrob Agents Chemother 4 7:1658-1664.

[35] Fenollar et al. (2003) Antimicrob Agents Chemother 47:1665-1671.

[36] WO03/035905.

[37] Balows (1974) Current techniques for antibiotic susceptibility testing. ISBN: 0398028869.

[38] Smaill (2000) Can J Gastroenterol14:871-875.

[39] Baquero (1990) Eur J Clin Microbial Infect Dis 9:492-495.

[40] Olsvik et al. (1994) Clin Microbial Rev 7:43-54.

[41] Bruno et al. (1996) J Mol Recognit 9:474-479.

[42] Zhang & Liu (2003) Expert Rev Mol Diagn 3:237-248.

[43] Hengen (1995) Trends Biochem Sci 20:372-3 73,

[44] Hill (2001) Expert Rev Mol Diagn 1:445-455.

[45] de Barbeyrac et al. (1995) Genitourin Med71 :382-386.

[46] Dean et al. (1998) J Clin Microbiol36:94-99.

[47] Puolakkainen et al. (1998) J Clin Microbio/36:1489-1493.

[48] Davis et al. (1998) Infect Dis Obstet Gynecol 6:57-60.

[49] Black et al. (2002) J Clin Microbio/40:3757-3763.

[50] Lin et al. (1999) Changgeng Yi Xue Za Zhi 22:204-211.

[51] Koumans et al. (2003) J Clin Microbio/41:1507-1511.

[52] Semeniuk et al. (2002) Diagn Microbial Infect Dis 42:43-51.

[53] Stary et al. (1998) J Clin Microbiol36:2666-2670.

[54] Templeton et al. (2001) Int J STD AIDS 12:793-796.

[55] Knox & Timms (1998) J Clin Microbiol36:3032-3039.

[56] Comanor et al. (2001) Am J Gastroentero/96:2968-2972.

[57] Oliveria et al. (2001) J Vet Diagn Invest 13:495-501.

[58] Bader et al. (2003) J Microbial Methods 52:209-220.

[59] Jurkevitch & Ramati (2000) FEMS Microbial Lett 184:265-271.

[60] Teng et al. (2000) J Clin Microbiol38:1672-1675.

[61] Takeshi et al. (1999) J Clin Microbiol37:4093-4098.

[62] Martineau et al. (1998) J Clin Microbiol36:618-623.

[63] Einsele et al. (1997) J Clin Microbio/35:1353-1360.

[64] Martineau et al. (1996) J Clin Microbiol34:2888-2893.

[65] Yamazaki & Nakamura (1995) Tuber Lung Dis 76:330-335.

[66] Picken (1992) J Clin Microbiol30:99-114.

[67] Frazier et al. (1992) Acta Viro/36:83-89.

[68] Gulledge et al. (2001) Appl Environ Microbio/67:4726-4733.

[69] Byl & Farmer (2001) page 150 of US Geological Survey Water-Resources Invenstigations Report Ol-401I (Ed. Kuniansky).

[70] International patent application PCT/GB2004/001517 (Acolyte Biomedica Limited).

1. A for analysing a biological sample, comprising the steps of:
(a) identifying from a patient sample, without prior culture, a micro-organism present within the sample; and
(b) determining the effect of one or more antimicrobial(s) on a micro-organism from the sample;
wherein step (a) is performed with a genotype-based technique and step (b) comprises assessing microbial numbers by measuring the nucleic acid content in a sample.

2. The process of claim 1, wherein step (a) involves a nucleic acid hybridisation assay.

3. The process of claim 1, wherein step (b) involves a nucleic acid hybridisation assay.

4. The process of claim 1, wherein step (a) and/or step (b) involves amplification of nucleic acid from the micro-organism.

5. The process of claim 4, wherein nucleic acid amplification uses the polymerase chain reaction.

6. The process of claim 4, wherein nucleic acid amplification uses primers which are specific to a micro-organism of interest.

7. The process of claim 1, wherein the micro-organism's DNA is analysed.

8. The process of claim 1, wherein the micro-organism's RNA is analysed.

9. The process of claim 7, wherein said DNA or RNA is a rRNA or rDNA.

10. The process of claim 1, wherein micro-organisms are extracted from the sample prior to step (a).

11. The process of claim 10, wherein micro-organisms are extracted by immunomagnetic separation.

12. The process of claim 1, wherein the antimicrobial(s) used in step (b) are selected based on the results of step (a).

13. The process of claim 1, wherein step (b) involves a comparison with data obtained in step (a).

14. The process of claim 1, wherein the micro-organism is a bacterium, a fungus, a parasite or a virus.

15. The process of claim 1, wherein the antimicrobial is an antibiotic, an antimycotic or an antiviral.
